(19) United States
(12) Patent Application Publication
(10) Pub. No.: US 2010/0168820 A1
(43) Pub. Date: Jul. 1, 2010

(54) AUTOMATIC THRESHOLD ASSESMENT UTILIZING PATIENT FEEDBACK

(75) Inventors: Jeremy Maniak, Edina, MN (US); Ralph Cardinal, White Bear Lake, MN (US); Hans Neisz, Coon Rapids, MN (US); Jason John Skubitz, Arden Hills, MN (US); Henry DeMorett, Bloomington, MN (US)

Correspondence Address: Leptos Biomedical, Inc.;c/o CPA GLOBAL, P.O. Box 52050, Minneapolis, MN, 52050, US

(73) Assignee: LEPTOS BIOMEDICAL INC., Fridley, MN (US)

(21) Appl. No.: 12/629,823

(22) Filed: Dec. 2, 2009

Related U.S. Application Data
(60) Provisional application No. 61/119,218, filed on Dec. 2, 2008.

(51) Int. Cl. A61N 1/08

(52) U.S. Cl. 607/63

(57) ABSTRACT

Methods, Implantable Pulse Generators (IPGs), and systems for stimulating a sympathetic nervous system nerve including automatically increasing the maximum stimulation current intensity over time. Some IPGS increase the current stimulation current maximum upon passage of an elapsed time or occurrence of a time of day. The current stimulation current maximum is the actual stimulation current in some methods and is a ramp maximum in other methods. The patient may interact with the IPG to indicate discomfort, resulting in a decrease in the current stimulation current maximum. In some methods, after receiving too many patient indications of discomfort, stimulation is stopped by the IPG.

### CROSS REFERENCE TO RELATED CASES

This application claims priority to U.S. Provisional patent 61,119,218, filed Dec. 2, 2008, which is herein incorporated by reference.

### FIELD

The disclosure relates to using patient feedback to optimize neuromodulation of the sympathetic nervous system.

### BACKGROUND

When peripheral nerves are electrically stimulated for the purpose of driving a therapeutic effect it is common that stimulation intensity is ramped up over a period of days or weeks in order to optimize therapeutic effect while minimizing patient discomfort. This is especially true for therapies that may require higher stimulation intensities in order to capture, or activate, smaller efferent fibers in the presence of larger afferent fibers. It is widely known that the stimulation intensity needed to capture a particular fiber is inversely proportional to the fiber diameter. In activating these smaller efferent fibers, the larger afferent fibers are activated. In some instances the stimulation can result in patient discomfort and low tolerability to the therapy. To solve this problem, intensity can be increased over longer periods of time via multiple clinical visits such that the activation of the larger afferent fibers is accommodated and the patient discomfort is greatly minimized.

This ramping process is very burdensome in its own right in that it requires multiple visits to the clinic for the patient along with additional clinician time. It would be desirable to implement this process in a manner that minimizes these costs while maintaining the benefit.

### SUMMARY

To solve this problem, a system was developed in which a chronically implanted pulse generator would autonomously proceed through an intensity ramping profile over a programmable period of days or weeks. Exemplary ramping stimulation patterns are described in U.S. Patent Application Number 2007/0203521, which is herein incorporated by reference in its entirety. The implanted pulse generator (i.e., that includes or is operably connected to an interface that allows a patient to control aspects of the stimulation) is then capable of receiving patient input from a patient programmer, or magnet, sound activated sensor, or tactile activated sensor (collectively herein after referred as a “patient intervention device,” or PID) that indicates patient discomfort at a particular level of stimulation. The IPG can utilize this patient feedback to adjust the intensity ramping profile in order to minimize patient discomfort while continuing to challenge the patient's tolerability threshold. The system may continue to incorporate patient feedback from the PID to customize the maximum stimulation intensity for the individual patient without clinician interaction. This autonomous intensity ramping profile can be terminated via either a clinician programmed duration or a consecutive number of patient interactions at a particular intensity level. When this optimal level of stimulation intensity is determined (i.e., maximum tolerable stimulation intensity) the implanted pulse generator may autonomously transition to a sequence of programmed therapeutic algorithms (stimulation patterns) in which this optimal intensity level is then utilized as the upper bound of intensity for these algorithms such that therapeutic effect is optimized while minimizing discomfort.

This intensity ramping profile may be programmed with a series of parameters (e.g. duration, maximum intensity allowed, # of patient interactions for termination, etc) such that the profile is maintained within predetermined safe limits throughout the autonomous process.

In some examples, a method for stimulating a sympathetic nerve using an IPG implanted in a subject is described. The implantable pulse generator (IPG) is programmed to stimulate a sympathetic nerve, such as the greater (GSN), lesser or least splanchnic nerve, using a maximum tolerable stimulation intensity level comprising a pulse width, current and frequency. The maximum tolerable stimulation intensity is used to refer to the stimulation intensity that a subject can tolerate over a duration of at least about a 24 hour period. It is understood that a subject may be able to sense the delivery of a stimulation pattern comprising a maximum tolerable stimulation intensity, however, the sensation will be tolerable. In humans the initial maximum tolerable stimulation intensity is typically established through interaction with a clinician and/or through a guided computer generated survey, wherein the patient is given the opportunity to provide input as to the tolerability of various stimulation intensities and the clinician or computer increases or decreases the stimulation intensity (i.e., by altering the pulse width, current or frequency) based upon the patient's input. Upon identification of the individual patient's maximum tolerable stimulation intensity, a stimulation pattern is initiated. The pattern is designed such that after an increment event has occurred the stimulation intensity is increased by a stimulation increase amount. One of ordinary skill in the art will appreciate that the stimulation increase amount can be any increase in energy that increases the stimulation intensity. For example, the increase in the stimulation increase amount can be caused by increasing one or more of the following: pulse width; frequency; and current. The methods described herein also include receiving a patient initiated signal from a PID. Exemplary patient initiated signals can be generated using PIDs such as a magnet, a patient programmer, patient movement or patient generated sound. In examples where sound is used to generate the signal, voice activation software and corresponding hardware can be used. In examples where pressure sensors are used the patient initiated signal can be derived from the application of pressure in the vicinity of the sensor. One of ordinary skill in the art will appreciate that the sensing of a signal from a PID can be accomplished in a component of the IPG, or in an independent sensor that is in communication with the IPG.

Upon receiving a patient initiated signal, the maximum tolerable stimulation intensity level is decreased. The decrease can be a preprogrammed increment of decrease or it can be established by additional patient initiated signals. Regardless of how the decrease is affected, the new, lower stimulation intensity becomes the maximum tolerable stimulation intensity and a stimulation pattern that comprises periodic increases to the maximum tolerable stimulation intensity can be initiated.

In some examples the increment event that triggers the stimulation increase amount can be a period of time such as, for example, about 1 hour, 5 hours, 10 hours, 15 hours, 20 hours, 48 hours, one week, two weeks, three weeks, one month or any other time increment that achieves the desired therapeutic benefit. In other examples, the increment event can be the occurrence of a time event such as one week of tolerated therapy or 2, 3, 4, or more weeks of tolerated therapy. In yet other examples the increment event is the reception by the IPG of an externally generated signal (i.e., from a clinician). The externally generated signal can be sensed by the IPG via any wireless technology, for example wireless internet technology, radiofrequency communication and the like.

The methods described herein include a patient initiated decrease in stimulation intensity during a stimulation pattern. The patient initiated decrease can be programmed such that each occurrence of a patient initiated decrease triggers the same amount of stimulation intensity decrease, however, in some examples the patient initiated decrease can vary. For instance, in reaction to a first patient initiated decrease in stimulation intensity a first decrease increment can occur, however, a subsequent second patient initiated decrease can trigger either a greater or lesser patient initiated decrease amount. For example, in instances where the patient is challenged to push the maximum tolerable threshold limit to as high as possible, the second patient initiated decrease amount can be lesser than the first decrease. Hence, offering some relief, but yet continuing to aggressively challenge the patient.

As previously mentioned, after a patient initiated decrease the stimulation pattern starts to periodically increase stimulation intensity again. In some instances the increase in stimulation intensity is equal to the decrease initiated by the patient. In other instances, the increase is a percentage of the decrease initiated by the patient. For example, the increase can be 1, 3, 5, 10, 20, 30, 40, 50, 60, 70, 80, or 90% of the amount of the decrease triggered by the patient. In additional examples, the stimulation intensity increase after a patient initiated decrease is less after multiple patient initiated decreases. In other words, if a patient continues to indicate that a maximum tolerable stimulation threshold is too much and therefore, initiates multiple decreases, the following stimulation intensity increases can be programmed to be smaller and smaller, thus allowing the patient to slow the stimulation challenge.

In some examples, upon receipt of a previously identified number of patient initiated decreases the electrical activation of the nerve is terminated. In some embodiments the patient initiated signal is such that it triggers a “pause” meaning that the stimulation intensity of the therapy is substantially decreased for a time period of about 30 minutes to about 6 hours, from about 1 to about 4 hours, or from about 1 hour to about 3 hours. After the pause time has elapsed, the stimulation pattern re-initiates using the maximum tolerable stimulation threshold that was being used prior to the patient initiated pause. The pause can be used by a patient whom, except for specific time period identified by the patient initiated pause, is generally tolerating the therapy at a specific maximum tolerable stimulation threshold. In some examples, the patient initiated pause can reduce stimulation to zero.

In additional examples, patients can initiate a signal to increase stimulation intensity (i.e., initiate a challenge themselves). One of ordinary skill in the art will appreciate that by using programming to challenge an individual patient's tolerance threshold and by allowing an individual patient to manually initiate a stimulation intensity increase allows for a customized therapy based upon individual pain/discomfort perception, as well as allows for adjustment of therapy to fit an individual's activities, and overall health status, without the need for visiting a clinic.

As described herein, the maximum tolerable stimulation intensity, stimulation intensity increase amount, stimulation intensity reduction amount, and other descriptions provided herein relating to stimulation intensities can be described as having pulse widths, currents and frequencies. One of ordinary skill in the art will appreciate that these stimulation intensities can be decreased or increased by altering one or more of these stimulation intensity characteristics. The pulse width can be changed in increments of 10 microseconds from about 80 microseconds to about 700 microseconds. In other words, the stimulation intensity can be increased by increasing the pulse width using a programmed stimulation pattern that periodically increases the pulse width by an increment of 20, 30, 40, 50, 60, 70, 80, 90, or 100 . . . microseconds. Conversely, the stimulation intensity can be decreased using similar increments. Similarly, the current can be changed in increments of about 0.10 mA from about 0.01 mA to about 10 mA. In other words, the stimulation intensity can be increased by increasing the current using a programmed stimulation pattern that periodically increases the current by an increment(s) of 0.01, 0.05, 0.10, 0.50, 0.60, 0.80, 0.10, 0.50, or 0.75 . . . mA. Conversely, the stimulation intensity can be decreased using similar increments. The frequency can also be increased or decreased depending upon the particular circumstance. The change can be in increments of 0.10 Hz from about 0.10 Hz to about 30 Hz. In other words, the stimulation intensity can be increased by increasing the frequency using a programmed stimulation pattern that periodically increases the frequency by an increment of 0.10, 0.20, 0.30, 0.50, 0.75, 0.80, 1.0, 1.5, or 2 . . . Hz. Conversely, the stimulation intensity can be decreased using similar increments. One of ordinary skill in the art will appreciate that larger or smaller increments can be used to change any of the stimulation intensity characteristics.

In some instances, the treatment also is titrated or modified based upon the relationship of a particular stimulation pattern to the level of a particular biomarker. In some examples, upon initiation of a patient initiated signal to either decrease stimulation or increase stimulation, a biomarker reading is triggered. For example, upon initiation of a patient initiated decrease signal a blood pressure or blood glucose level is taken. This information can be used to alert a clinician to a possible unsafe status and/or can be used to optimize or establish therapeutic targets. Other biomarkers that can be used include temperature, heart rate, satiety signaling molecules, hormones, lipolysis markers and diabetic markers including for example insulin and glucagon, nerve recording (EGM) changes, transthoracic impedance, and other EGM recordings of physiologic parameters. For example, biomarkers such as glucose, catecholamines, blood pressure, insulin, glucagon, incretins, free fatty acids and glycerol can be measured and directly associated with the patient initiated signal and used to re-evaluate therapy. In additional examples, biomarker readings can also be taken on a more scheduled basis throughout therapy.

Methods of treating insulin resistance and/or T2D are also disclosed. These methods include selecting a subject that has insulin resistance, initiating an electrical stimulation pattern wherein the pattern includes a maximum tolerable stimulation intensity and receiving a patient initiated signal from a PID that interrupts the stimulation pattern and changes the characteristics of the maximum tolerable stimulation intensity to decrease the sensation caused by the electrical stimulation pattern. The methods also include subsequently automatically increasing the stimulation intensity.

Subjects selected for insulin resistance and/or T2D treatment can be selected using any method known in the art. For example, subjects can be chosen based upon their HbA1c, fasting glucose and/or fasting insulin levels. Methods of identifying such subjects are well known in the art. For example, the Homeostatic Model Assessment (HOMA), or the Quantitative Insulin Sensitivity Check Index (QUICKI) methods can be used. Both employ fasting insulin and glucose levels to calculate insulin resistance, and both correlate reasonably with the results of more research oriented tests that are not clinically practical. It is believed that patients treated using these methods can reduce their insulin resistance and that in some instances such reduction will not be accompanied by a significant loss in overall weight.

Additional methods that are provided herein include methods of reducing central adiposity. Subjects selected for such therapy can be selected using any method known in the art. For example, methods such as dual-energy x-ray absorptiometry (DEXA), circumference measurement, or computed axial tomography (CT) can be used. These subjects are started on an electrical stimulation pattern that periodically increases the maximum tolerable stimulation intensity and are also provided with a PID to allow for patient controlled increase or decrease of the stimulation intensity. It is believed that these patients can reduce their central adiposity and that, in some instances, such reduction will not be accompanied by a proportional loss in overall weight.

### DESCRIPTION OF DRAWINGS

FIG. 1 is a diagrammatic view of an efferent autonomic nervous system of a human.

FIG. 2 is a diagrammatic view of a sympathetic nervous system anatomy.

FIG. 3 is an elevation view of the splanchnic nerves and celiac ganglia.

FIG. 4 is a schematic view of an exemplary stimulation pattern.

FIG. 5 is a schematic diagram of an exemplary ramp-cycling treatment algorithm.

FIG. 6 shows a portion of the ramp-cycling treatment algorithm of FIG. 5 in more detail.

FIG. 7 shows a more detailed view of a portion of the exemplary stimulation pattern of FIG. 6.

FIG. 8 is a state diagram of logic executed in the IPG of one embodiment of the invention.

FIG. 9 is a photograph of a handheld patient programmer device used by a patient for interfacing with an IPG and can be used in challenge mode.

FIG. 10 is a screenshot of a screen used in a clinical programming device to configure the challenge mode parameters in the IPG, typically in a physician's office.

FIG. 11 is a diagram showing an exemplary implant position.

FIG. 12 is an image showing an exemplary IPG.

FIG. 13 is an image showing an exemplary electrode.

### DETAILED DESCRIPTION

The invention includes a method for treating obesity, metabolic syndrome, T2D, or other disorders (collectively referred to as “target disorders”) by electrically activating the sympathetic nervous system with an electrode on or near a nerve, or with a wireless electrode inductively coupled with a radiofrequency field. In some embodiments, obesity (or the other disorders mentioned above) can be treated by activating the efferent sympathetic nervous system, thereby increasing energy expenditure and reducing food intake. Stimulation can be accomplished using a radiofrequency pulse generator and electrodes implanted near, or attached to, various areas of the sympathetic nervous system, such as the sympathetic chain ganglia, the splanchnic nerves (greater, lesser, least), or the peripheral ganglia (e.g., celiac, mesenteric). In some embodiments, the obesity therapy will employ electrical activation of the sympathetic nervous system that innervates the digestive system, adrenals, and abdominal adipose tissue, such as the splanchnic nerves or celiac ganglia. Afferent stimulation can also be accomplished to provide central nervous system satiety. Afferent stimulation can occur by a reflex arc secondary to efferent stimulation. In some embodiments, both afferent and efferent stimulation can be achieved.

This method of target disorder treatment may reduce food intake by a variety of mechanisms, including, for example, general increased sympathetic system activation and increasing plasma glucose levels upon activation. Satiety may be produced through direct effects on the pylorus and duodenum that cause reduced peristalsis, stomach distention, and/or delayed stomach emptying. In addition, reducing ghrelin secretion and/or increasing PYY secretion may reduce food intake. The method can also cause weight loss by reducing food absorption, presumably through a reduction in secretion of digestive enzymes and fluids and changes in gastrointestinal motility. Increased stool output, increased PYY concentrations (relative to food intake), and decreased ghrelin concentrations (relative to food intake) may be the result of splanchnic nerve stimulation according to the stimulation parameters disclosed herein.

This method of target disorder treatment may also increase energy expenditure by causing catecholamine, cortisol, and dopamine release from the adrenal glands. The therapy can be titrated to the release of these hormones. Fat and carbohydrate metabolism, which are also increased by sympathetic nerve activation, may accompany the increased energy expenditure. Other hormonal effects induced by this therapy may include reduced insulin secretion. Alternatively, this method may be used to normalize catecholamine levels, which are reduced with weight gain.

Electrical sympathetic activation for treating T2D can be also accomplished without cause a rise in Mean Arterial Blood Pressure (MAP). Surprisingly, electrical sympathetic activation can be also be used to treat T2D and co-morbidities without causing significant weight loss. For example, a patient that is treated with sympathetic simulation, as described herein, can be treated such that their hemoglobin A1c (HbA1c) decreases over time, however, their weight remains substantially the same. For example, in some embodiments a patient's weight is within 5% of their pretreatment weight after six months of therapy. In other embodiments a patient's weight is within 4%, 3%, or 2% of their pretreatment weight after 6 months of therapy.

In some examples, a patient's HbA1c decreases by 0.5%/six months of therapy, in other examples the patient's HbA1c decreases by at least 1%, 1.1%, 1.3%, 1.5%, 1.7% or 2% per 6 months of treatment. One of ordinary skill in the art will appreciate that the amount of circulating HbA1c relates to the patient's blood glucose level overtime. Therefore, when stimulation patterns that reduce HbA1C are used it is also expected that the patient's average blood glucose concentration will be reduced.

In some instances the overall weight of the patient remains substantially the same as described herein, however, their visceral fat mass reduces and their lean muscle mass remains the same or increases on a percentage basis. It is believed that this is due, in part, to the stimulation of the visceral fat pads, as well as an overall increase in localized lipolysis.

As mentioned above, in some instances the improvements in glycemic control, as reflected by a significant reduction in HbA1c, are more significant than the limited change in weight would suggest. It is believed that this is accomplished because various stimulation patterns trigger GSN activity that causes at least one of the following metabolic mechanisms.

The first metabolic mechanism that is believed to result from some GSN stimulation patterns described herein is targeted reduction of visceral fat depots via the stimulation of lipolytic activity of the visceral fat pads combined with slight caloric reduction due to the satiety effects of stimulation. This yields a slight caloric deficit that is utilized to preferentially reduce the visceral fat stores. The preferential reduction in visceral fat results in an improvement in the secretion of adipokines and cytokines that have negative impact on hepatic and peripheral insulin sensitivity along with beta cell function exceeding the weight change alone since visceral fat depots contribute more significantly to these negative impacts than other fat depots. The preferential reduction in visceral fat also results in a preferential reduction in Non-Esterified Fatty Acids (NEFA) circulation. Chronic increases in NEFA circulation are also causally linked to decreased hepatic and peripheral insulin sensitivity. By preferentially targeting visceral fat stores GSN stimulation targets at least two of the most significant causes of insulin resistance, a precursor to T2D.

The second metabolic mechanism that is believed to result from some exemplary GSN simulation patterns is the secretion of incretins from K and L cells from the gastrointestinal tract (GI) tract as a result of GSN modulation. By increasing the level of incretins, such as GLP-1 and/or GIP, beta cell mass and function can be improved along with improving insulin secretion in a glucose dependent manner, thus limiting the risks of hypoglycemia.

The third metabolic mechanism that is believed to result from some exemplary GSN stimulation patterns is through reduced absorption of carbohydrates and fat during a meal via alterations in gastric motility and absorption as a result of GSN modulation. Reducing the absorption of carbohydrates during feeding improves portal and systemic hyperglycemia resulting from a carbohydrate or glucose load. Reductions in the dynamic range of glucose during meals results in improvements in the insulin requirements and as such enables improvements in beta cell function.

Electrical sympathetic activation for treating obesity may be accomplished without causing a rise in MAP. This can be achieved by using an appropriate stimulation pattern with a relatively short signal-on time (or “on period”) followed by an equal or longer signal-off time (or “off period”). In certain embodiments, this may be achieved by using an appropriate stimulation pattern with a continuous signal-on time, wherein the signal-on time is comprised of a relatively short suprathreshold period, during which the energy delivered to a nerve or nerve fiber group meets or exceeds a threshold for exciting that nerve or nerve fiber group, followed by an equal or longer subthreshold period, during which the energy delivered to the nerve or nerve fiber is below the threshold. During activation therapy, a sinusoidal-like fluctuation in the MAP can occur with an average MAP that is within safe limits. Alternatively, an alpha sympathetic receptor blocker, such as prazosin, can be used to blunt the increase in MAP.

Electrical sympathetic activation for treating obesity may be accomplished without permitting a regain of the previously lost weight during the period in which the stimulator is turned off. This can be achieved by using a stimulation time period comprising consecutive periods in which each period has a stimulation intensity greater than the preceding stimulation period. In some embodiments, the stimulation intensity during the first stimulation period is set at about the maximum tolerable stimulation intensity. The consecutive stimulation periods are followed by a no-stimulation time period in which the stimulator remains off or emits only a subthreshold amount of power.

Electrical sympathetic activation for treating obesity may also be accomplished without permitting a regain of the previously lost weight during a subthreshold period. This may be achieved by using a stimulation time period comprising consecutive suprathreshold periods in which each period has a stimulation intensity greater than the preceding suprathreshold stimulation period. In some embodiments, the stimulation intensity during the first suprathreshold stimulation period is set at the maximum tolerable stimulation intensity. The consecutive suprathreshold stimulation periods are followed by a subthreshold time period.

Treatment effectiveness may be increased if the stimulation patterns are adjusted to prevent the body from compensating for the stimulation. In certain embodiments, this can be achieved by changing the maximum tolerable stimulation intensity reached during consecutive groups of stimulation periods, even in the absence of a no-stimulation time period.

A dynamic stimulation technique using ramp-cycling can be used on cranial nerves, the spinal cord, and/or other peripheral nerves, including those in the autonomic system and other motor and sensory nerves.

As previously mentioned, electrical sympathetic activation can be titrated to the plasma level of catecholamines achieved during therapy. This would allow the therapy to be monitored and safe levels of increased energy expenditure to be achieved. The therapy can also be titrated to plasma ghrelin levels or PYY levels.

As used herein, electrical “modulation” of a nerve (or nerve fiber group) can include excitation (elicitation of one or more action potentials), inhibition, or a combination of these. Electrical “activation” generally includes excitation, but can also include inhibition and/or periods of little or no energy delivery to the nerve (or nerve fiber). Electrical modulation (inhibition or activation) of the sympathetic nerves can also be used to treat other eating disorders such as anorexia or bulimia. For example, inhibition of the sympathetic nerves can be useful in treating anorexia. Electrical modulation of the sympathetic nerves may also be used to treat gastrointestinal diseases such as peptic ulcers, esophageal reflux, gastroparesis, and irritable bowel. For example, stimulation of the splanchnic nerves that innervate the large intestine may reduce the symptoms of irritable bowel syndrome, characterized by diarrhea. Pain may also be treated by electric nerve modulation of the sympathetic nervous system, as certain pain neurons are carried in the sympathetic nerves. This therapy may also be used to treat T2D. These conditions can require varying degrees of inhibition or stimulation.

Attendant or contributing conditions of obesity, metabolic syndrome, and T2D can include, but are not limited to, obesity, dyslipidemia, hypertension, hyperinsulinemia, elevated plasma glucose levels, hyperglycemia, a decreased lean muscle mass fraction of total body mass, an increased visceral or abdominal fat fraction of total body mass, or high blood pressure. Dyslipidemia can include, but is not limited to, elevated levels of total cholesterol, elevated levels of triglycerides, elevated levels of LDL, or decreased levels of HDL. One of ordinary skill in the art will understand that ameliorating or treating an attendant or contribution condition of T2D can be equivalent to ameliorating or treating an attendant condition of metabolic syndrome.

As discussed above, the indicators or attendant or contributing conditions of metabolic syndrome include obesity, and particularly obesity around the waist. A waistline of 40 inches or more for men and 35 inches or more for women would qualify. Another attendant or contributing condition is high blood pressure such as a blood pressure of 130/85 mm Hg or greater. Yet another attendant or contributing condition is one or more abnormal cholesterol levels including a high density lipoprotein level (HDL) less than 40 mg/dl for men and under 50 mg/dl for women. A triglyceride level above 150 mg/dl may also be an indicator. Finally, a resistance to insulin is an indicator of metabolic syndrome which may be indicated by a fasting blood glucose level greater than 100 mg/dl. As such, treatment of one, two, three or more of these indicators of metabolic syndrome may be effective in treatment of metabolic syndrome as it is the conglomeration of several or all of these conditions that results in metabolic syndrome.

Neural stimulation has been used for treatment of various medical conditions including pain management, tremor and the like. Neural stimulation has also been shown to be useful in treating obesity in mammals as well as for regulating certain hormone levels. Embodiments are directed to systems and methods of neural stimulation or modulation including activation and inhibition for treating metabolic syndrome or its attendant or contributing conditions either individually or in combination. Certain embodiments disclosed herein are directed to systems and methods of neural stimulation or modulation. The modulation of nerve tissues such as autonomic nerve tissue including central and peripheral, sympathetic and parasympathetic, may be used to achieve a desired physiological result or treatment of various medical conditions. Specific nerve tissue such as the splanchnic nerve, vagus nerve, stellate ganglia and the like may be modulated in order to achieve a desired result.

The human nervous system is a complex network of nerve cells, or neurons, found centrally in the brain and spinal cord and peripherally in the various nerves of the body. Neurons have a cell body, dendrites and an axon. A nerve is a group of neurons that serve a particular part of the body. Nerves can contain several hundred neurons to several hundred thousand neurons. Nerves often contain both afferent and efferent neurons. Afferent neurons carry signals back to the central nervous system and efferent neurons carry signals to the periphery. A group of neuronal cell bodies in one location is known as a ganglion. Electrical signals are conducted via neurons and nerves. Neurons release neurotransmitters at synapses (connections) with other nerves to allow continuation and modulation of the electrical signal. In the periphery, synaptic transmission often occurs at ganglia.

The electrical signal of a neuron is known as an action potential. Action potentials are initiated when a voltage potential across the cell membrane exceeds a certain threshold. This action potential is then propagated down the length of the neuron. The action potential of a nerve is complex and represents the sum of action potentials of the individual neurons in it. Neurons can be myelinated and unmyelinated and of large axonal diameter and small axonal diameter. In general, the speed of action potential conduction increases with myelination and with neuron axonal diameter. Accordingly, neurons are classified into type A, B and C neurons based on myelination, axon diameter, and axon conduction velocity. In terms of axon diameter and conduction velocity, A is greater than B which is greater than C.

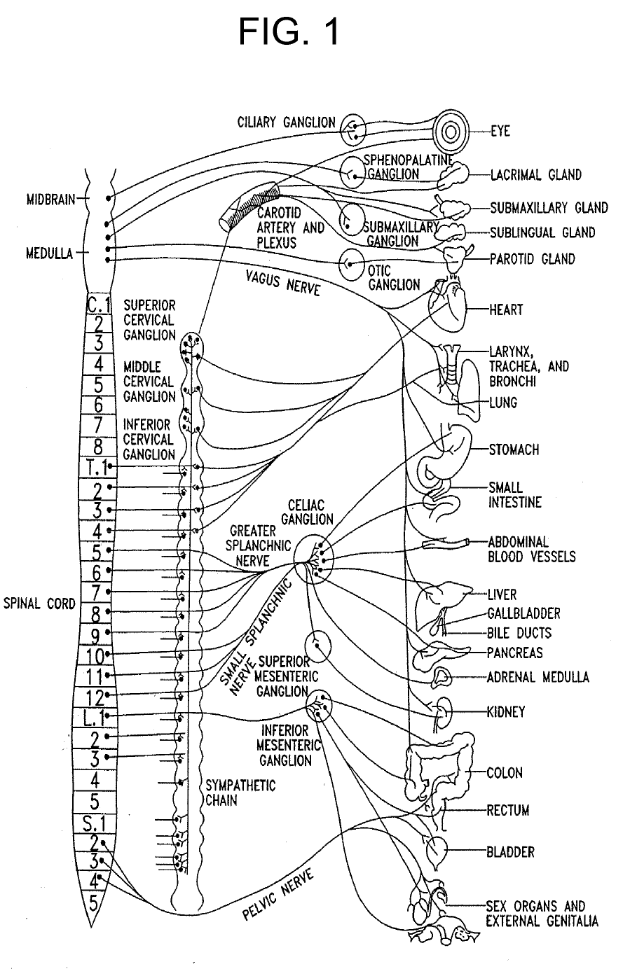

The autonomic nervous system is a subsystem of the human nervous system that controls involuntary actions of the smooth muscles (blood vessels and digestive system), the heart, and glands, as shown in FIG. 1. The autonomic nervous system is divided into the sympathetic and parasympathetic systems. The sympathetic nervous system generally prepares the body for action by increasing heart rate, increasing blood pressure, and increasing metabolism. The parasympathetic system prepares the body for rest by lowering heart rate, lowering blood pressure, and stimulating digestion.

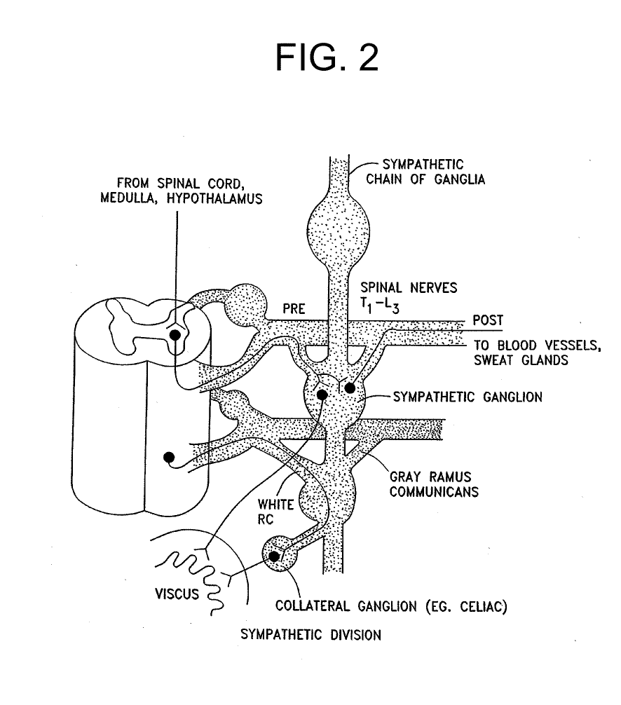

The hypothalamus controls the sympathetic nervous system via descending neurons in the ventral horn of the spinal cord, as shown in FIG. 2. These neurons synapse with preganglionic sympathetic neurons that exit the spinal cord and form the white communicating ramus. The preganglionic neuron will either synapse in the paraspinous ganglia chain or pass through these ganglia and synapse in a peripheral, or collateral, ganglion such as the celiac or mesenteric. After synapsing in a particular ganglion, a postsynaptic neuron continues on to innervate the organs of the body (heart, intestines, liver, pancreas, etc.) or to innervate the adipose tissue and glands of the periphery and skin. Preganglionic neurons of the sympathetic system can be both small-diameter unmyelinated fibers (type C-like) and small-diameter myelinated fibers (type B-like). Postganglionic neurons are typically unmyelinated type C neurons.

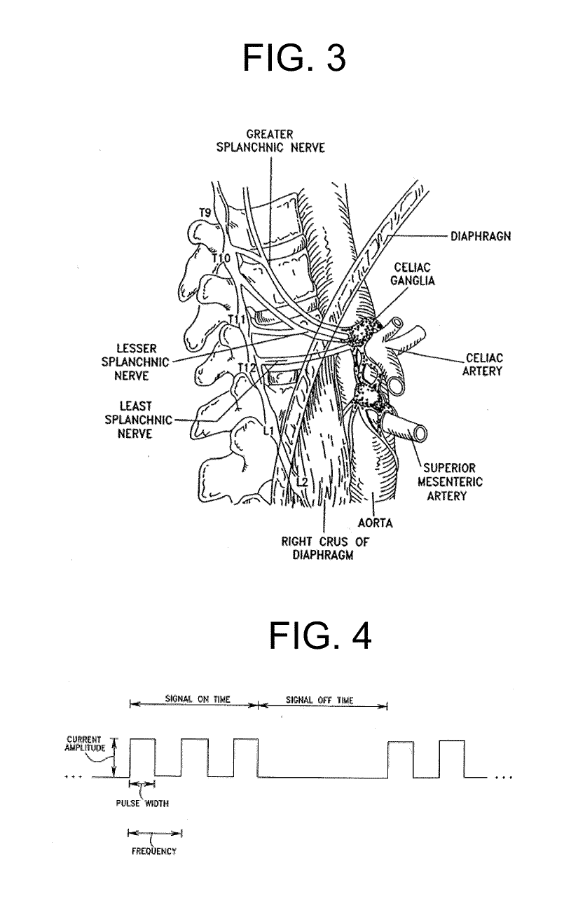

Several large sympathetic nerves and ganglia are formed by the neurons of the sympathetic nervous system as shown in FIG. 3. The greater splanchnic nerve (GSN) is formed by efferent sympathetic neurons exiting the spinal cord from thoracic vertebral segment numbers 4 or 5 (T4 or T5) through thoracic vertebral segment numbers 9 or 10 or 11 (T9, T10, or T11). The lesser splanchnic (lesser SN) nerve is formed by preganglionic fibers sympathetic efferent fibers from T10 to T12 and the least splanchnic nerve (least SN) is formed by fibers from T12. The GSN is typically present bilaterally in animals, including humans, with the other splanchnic nerves having a more variable pattern, present unilaterally or bilaterally and sometimes being absent. The splanchnic nerves run along the anterior lateral aspect of the vertebral bodies and pass out of the thorax and enter the abdomen through the crus of the diaphragm. The nerves run in proximity to the azygous veins. Once in the abdomen, neurons of the GSN synapse with postganglionic neurons primarily in celiac ganglia. Some neurons of the GSN pass through the celiac ganglia and synapse on in the adrenal medulla. Neurons of the lesser SN and least SN synapse with post-ganglionic neurons in the mesenteric ganglia.

Postganglionic neurons, arising from the celiac ganglia that synapse with the GSN, innervate primarily the upper digestive system, including the stomach, pylorus, duodenum, pancreas, and liver. In addition, blood vessels and adipose tissue of the abdomen are innervated by neurons arising from the celiac ganglia/greater splanchnic nerve. Postganglionic neurons of the mesenteric ganglia, supplied by preganglionic neurons of the lesser and least splanchnic nerve, innervate primarily the lower intestine, colon, rectum, kidneys, bladder, and sexual organs, and the blood vessels that supply these organs and tissues.

In the treatment of obesity, some embodiments of treatment involve electrical activation of the greater splanchnic nerve of the sympathetic nervous system. Unilateral activation may be utilized, although bilateral activation may also be utilized. The celiac ganglia can also be activated, as well as the sympathetic chain or ventral spinal roots.

Electrical nerve modulation (nerve activation, stimulation, and/or inhibition) is accomplished by applying an energy signal (pulse) at a certain frequency to the neurons of a nerve (nerve stimulation). The energy pulse causes depolarization of neurons within the nerve above the activation threshold resulting in an action potential. The energy applied is a function of the current (or voltage) amplitude and pulse width or duration. Activation or inhibition can be a function of the frequency of the energy signal, with low frequencies on the order of 1 to 50 Hz resulting in activation of a nerve for some embodiments and high frequencies greater than 100 Hz resulting in inhibition of a nerve for some embodiments. Inhibition can also be accomplished by continuous energy delivery resulting in sustained depolarization. Different neuronal types may respond to different energy signal frequencies and energies with activation or inhibition.

Each neuronal type (i.e., type A, B, or C neurons) has a characteristic pulse amplitude-duration profile (energy pulse signal or stimulation intensity) that leads to activation. The stimulation intensity can be described as the product of the current amplitude and the pulse width. Myelinated neurons (types A and B) can be stimulated with relatively low current amplitudes, on the order of 0.1 to 5.0 mA, and short pulse widths, on the order of about 50 μsec to about 200 μsec. Unmyelinated type C fibers typically require longer pulse widths on the order of about 300 μsec to about 1,000 μsec and higher current amplitudes for stimulation. Thus, in certain embodiments, the stimulation intensity for efferent activation of a nerve may be in the range of about 0.005 mA-msec to about 5.0 mA-msec. In certain embodiments, the stimulation intensity for efferent activation of a nerve may be in the range of about 0.001 mA-msec to about 10.0 mA-msec.

The greater splanchnic nerve also contains type A fibers. These fibers can be afferent and sense the position or state (contracted versus relaxed) of the stomach or duodenum. Stimulation of A fibers may produce a sensation of satiety by transmitting signals to the hypothalamus. They can also participate in a reflex arc that affects the state of the stomach. Activation of both A and B fibers can be accomplished because stimulation parameters that activate efferent B fibers will also activate afferent A fibers. Activation of type C fibers may cause both afferent an efferent effects, and may cause changes in appetite and satiety via central or peripheral nervous system mechanisms.

Various stimulation patterns, ranging from continuous to intermittent, may be utilized for various embodiments. In certain embodiments, information related to a stimulation pattern may be stored in a storage module. For example, stimulation pattern data may be stored in volatile memory, such as random access memory (“RAM”), or in non-volatile memory, such as a hard disk drive or flash drive.

With intermittent stimulation of nerves, an energy signal is delivered to a nerve or nerve tissue for a period of time at a certain frequency during the signal on-time as shown in FIG. 4. The signal on-time may be followed by a period of time with no energy delivery, referred to as a signal-off time. In certain embodiments, the signal on-time comprises a suprathreshold period, during which the energy delivered to a nerve or nerve fiber group (containing one or more nerve fibers) meets or exceeds a threshold for exciting (i.e., eliciting an action potential from) that nerve or nerve fiber group. In certain embodiments, the signal on-time comprises a subthreshold period, during which the energy delivered to the nerve or nerve fiber is below a threshold for exciting (i.e., eliciting an action potential from) that nerve (or nerve fiber group). Such a subthreshold period may comprise a period of no (or about zero) energy delivery, or an amount of energy greater than zero but less than that needed for exciting the nerve (or fiber). On average, the energy or power delivered to a nerve during a subthreshold period is greater than zero, even if there are one or more brief periods of zero-energy delivery. In certain embodiments as described herein using a signal-on time and signal-off time, a signal-on time may consist of a continuous or nearly continuous suprathreshold period. Consequently, as described herein, the effects of certain embodiments that use a signal-on time and signal-off time may be accomplished using properly configured subthreshold and suprathreshold periods during a continuous or nearly continuous signal-on time.

The ratio of the signal on-time to the sum of the signal on-time plus the signal off time is referred to as the duty cycle and it can, in some embodiments, range from about 1% to about 100%. The ratio of the suprathreshold period to the sum of the suprathreshold period plus the subthreshold period may also be referred to as a duty cycle and it can, in some embodiments, range from about 1% to about 100%. “Duty cycle” in the first definition above may be clarified as the ratio of the suprathreshold period to the sum of the suprathreshold period plus the subthreshold period (i.e., the total on-time) plus the off-time (i.e., the ratio of the suprathreshold period to the sum of the on-time and off-time). Such a duty cycle can, in some embodiments, also range from about 1% to about 100%. Peripheral nerve stimulation is commonly conducted at nearly a continuous, or 100%, duty cycle. However, an optimal duty cycle for splanchnic nerve stimulation to treat obesity may be less than 75% in some embodiments, less than 50% in some embodiments, or even less than 30% in certain embodiments. This may reduce problems associated with muscle twitching as well as reduce the chance for blood pressure or heart rate elevations caused by the stimulation energy. The on-time may also be important for splanchnic nerve stimulation in the treatment of obesity. Because some of the desired effects of nerve stimulation may involve the release of hormones, on-times sufficiently long enough to allow plasma levels to rise are important. Also, gastrointestinal effects on motility and digestive secretions take time to reach a maximal effect. Thus, an on-time of approximately 15 seconds, and sometimes greater than 30 seconds, may be used.

Superimposed on the duty cycle and signal parameters (frequency, on-time, mAmp, and pulse width) are treatment parameters. Therapy may be delivered at different intervals during the day or week, or continuously. Continuous treatment may prevent binge eating during the off therapy time. Intermittent treatment may prevent the development of tolerance to the therapy. A desirable intermittent therapy embodiment may be, for example, 18 hours on and 6 hours off, 12 hours on and 12 hours off, 3 days on and 1 day off, 3 weeks on and one week off or a another combination of daily or weekly cycling. Alternatively, treatment may be delivered at a higher interval rate, say, about every three hours, for shorter durations, such as about 2 minutes to about 30 minutes. The treatment duration and frequency may be tailored to achieve a desired result. Treatment duration for some embodiments may last for as little as a few minutes to as long as several hours. Also, splanchnic nerve activation to treat obesity may be delivered at daily intervals, coinciding with meal times. Treatment duration during mealtime may, in some embodiments, last from 1 hour to about 3 hours and start just prior to the meal or as much as an hour before.

Efferent modulation of the GSN may be used to control gastric distention/contraction and peristalsis. Gastric distention or relaxation and reduced peristalsis can produce satiety or reduced appetite for the treatment of obesity. These effects may be caused by activating efferent B or C fibers at moderate to high intensities, such as about 1.0 mA to about 5.0 mA current amplitude and about 0.15 to about 1.0 millisecond pulse width and higher frequencies of about 10 Hz to about 20 Hz. Gastric distention may also be produced via a reflex arc involving the afferent A fibers. Activation of A fibers may cause a central nervous system mediated reduction in appetite or early satiety. These fibers may be activated at the lower range of stimulation intensity, for example about 0.15 msec to about 0.30 msec pulse width and about 0.1 to about 1.0 mA current amplitude and higher range of frequencies given above. Contraction of the stomach can also reduce appetite or cause satiety. Contraction can be caused by activation of C fibers in the GSN. Activation of C fibers may also play a role in centrally mediated effects. Activation of these fibers is accomplished at higher stimulation intensities, for example about 2 to about 5 times those of B and A fibers.

It should be noted that the current amplitude of a stimulation signal may also vary depending on the type of energy delivery module (such as an electrode) used. A helical electrode that has intimate contact with the nerve will have a lower amplitude than a cylindrical electrode that may reside millimeters away from the nerve. In general, the current amplitude used to cause stimulation is proportional to 1/(Radial Distance From Nerve) 2. The pulse width can remain constant or can be increased to compensate for the greater distance. The stimulation intensity would be adjusted to activate the afferent/efferent B or C fibers depending on the electrodes used. Using the muscle [symbol] twitching threshold prior to habituation can help guide therapy, given the variability of contact/distance between the nerve and electrode.

Weight loss or other therapeutic benefits (i.e., treating T2D, insulin resistance, and metabolic syndrome) induced by electrical activation of the splanchnic nerve may be amplified by providing dynamic nerve modulation or stimulation. Dynamic stimulation refers to changing the values of stimulation signal intensity, stimulation frequency and/or the duty cycle parameters during treatment. The stimulation intensity, stimulation frequency and/or duty cycle parameters may be changed independently, or they may be changed in concert. One parameter may be changed, leaving the others constant; or multiple parameters may be changed approximately concurrently. The stimulation intensity, stimulation frequency and/or duty cycle parameters may be changed at regular intervals, or they may be ramped up or down substantially continuously. The stimulation intensity, stimulation frequency and/or duty cycle parameters may be changed to preset values, or they may be changed to randomly generated values. In some embodiments, the changes in the stimulation signal parameters are altered through an automated process, for example, a programmable pulse generator. When random changes in the stimulation signal parameter or parameters are desired, those changes may be generated randomly by a pulse generator. One advantage of dynamic stimulation is that the patient's body is unable, or at least less able, to adapt or compensate to the changing stimulation than to a constant or regular pattern of stimulation.

Therapeutic benefits induced by electrical activation of the splanchnic nerve may be improved by providing intermittent therapy, or intervals of electrical stimulation followed by intervals of no stimulation. For example, data shows that after an interval of stimulation, weight loss can be accelerated by turning the stimulation signal off. This is directly counter to the notion that termination of therapy would result in a rebound phenomenon of increased food intake and weight gain. This data also indicates that a dynamic, or changing, stimulation intensity (e.g., increasing or decreasing daily) produces a more pronounced weight loss than stimulation at a constant intensity. This intermittent therapy, coupled with a dynamic or changing stimulation intensity, is called the ramp-cycling technique, and ramp cycling is one subset of the dynamic stimulation techniques described herein. Given these findings, several dosing strategy embodiments are described below.

These treatment algorithm embodiments (sometimes referred to as stimulation patterns) are derived from studies involving canines. The muscle twitch threshold (which is similar to the maximum tolerable stimulation intensity in other subjects) is determined after adequate healing time post implant has elapsed which is typically about 2 to about 6 weeks. In certain embodiments, this threshold may range from about 0.125 mA-msec to about 0.5 mA-msec. The stimulation intensity is increased daily over about 1 to about 2 weeks, allowing some or complete habituation of muscle twitching to occur between successive increases, until an intensity of about 8 times to about 10 times the signal intensity of the muscle twitch threshold is achieved, for example about 1.0 mA-msec to about [symbol] 5.0 mA-msec. In certain embodiments, the stimulation intensity and/or the stimulation frequency is increased until an intensity of about 2 times the signal intensity of the muscle twitch threshold is achieved. In certain embodiments, the stimulation intensity is increased until an intensity of about 4 times the signal intensity of the muscle twitch threshold is achieved. In certain embodiments, the stimulation intensity is increased until an intensity of about 6 times the signal intensity of the muscle twitch threshold is achieved. During this period, a rapid decline in body weight and food intake is generally observed.

After the initial weight loss period, a transition period is observed over about 1 to about 4 weeks in which some lost weight may be regained. Subsequently, a sustained, gradual reduction in weight and food intake occurs during a prolonged stimulation phase of about 4 weeks to about 8 weeks. After this period of sustained weight loss, the stimulation may be terminated, which is again followed by a steep decline in weight and food intake, similar to the initial stimulation intensity ramping phase. The post-stimulation weight and food decline may last for about 1 week to about 4 weeks, after which the treatment algorithm may be repeated to create a therapy cycle, or intermittent treatment interval, that results in sustained weight loss. The duty cycle during this intermittent therapy may range from about 20% to about 50% with stimulation on-times of up to about 15 seconds to about 60 seconds. This intermittent therapy not only increases the weight loss effectiveness, but also extends the battery life of an implanted device or reduces energy consumption for a non-implanted pulse generator.

In another intermittent therapy treatment algorithm embodiment, therapy cycling occurs during about a 24 hour period. In this algorithm, the stimulation signal intensity is maintained at about 1 times to about 3 times the muscle twitch threshold for a period of about 12 hours to about 18 hours. In certain embodiments, the stimulation signal intensity may be increased gradually (e.g., each hour) during a first stimulation interval. In certain embodiments, the stimulation signal intensity may be increased at other intervals during a first stimulation interval. The stimulation is subsequently terminated or reduced to a subthreshold level for about 6 hours to about 12 hours. In certain embodiments, the stimulation signal intensity may be gradually decreased during a second interval back to a signal intensity substantially at the muscle twitch threshold level. Due to this sustained or accelerating effect that occurs even after cessation of stimulation, the risk of binge eating and weight gain during the off period or declining stimulation intensity period is minimized.

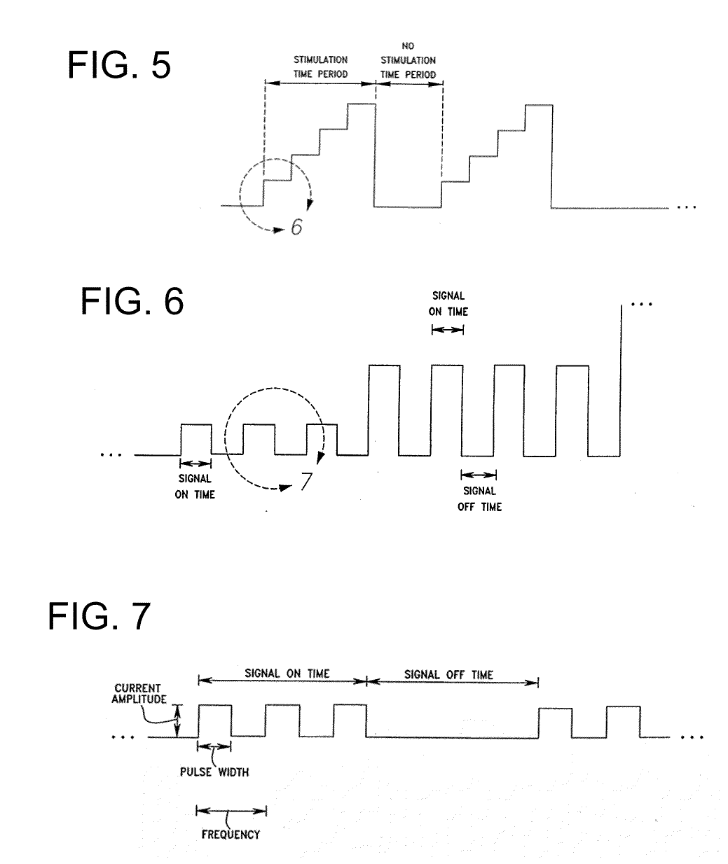

Certain embodiments utilize the ramp-cycling therapy or the ramp-cycling technique. One embodiment of the ramp-cycling technique is shown schematically in FIGS. 5-7. FIG. 5 has a longer time scale than FIG. 6, which in turn has a longer time scale than FIG. 7. FIG. 5 shows the main features of one embodiment of the ramp-cycling technique. Each period of the cycle includes a stimulation time period (or stimulation period) and a no-stimulation time period (or no-stimulation period). The stimulation time period may be referred to as a first time period, an interval of electrical stimulation, an interval of stimulation, a stimulation intensity ramping phase, or a stimulation interval. In certain embodiments, the stimulation time period may include on-times, off-times, suprathreshold periods, and subthreshold periods. The no-stimulation time period may be referred to as a second time period, an interval in which the device is off or delivering low power, an interval of no stimulation, or a declining stimulation intensity period. In certain embodiments, the no-stimulation time period may include one or more subthreshold periods. The stimulation time period and no-stimulation time period should not be confused with the stimulation on-time, signal on-time (or on-period or on-time), or the signal off-time (or off-period or off-time) which are terms describing the parameters of the duty cycle and shown in FIGS. 6 and 7. The stimulation time period further comprises portions or consecutive intervals.

In some embodiments of the ramp-cycling version of intermittent therapy, the stimulation time period comprises at least two portions having different stimulation intensities. The portions may also be referred to as consecutive intervals. In certain embodiments, the stimulation intensity of each portion may be greater than the stimulation intensity of the previous portion. The multiple portions of such an embodiment are represented by the stimulation time period's step-like structure as shown in the embodiment in FIG. 5. In certain embodiments, the increase in stimulation intensity is approximately continuous over the entire stimulation time period, rather than increasing in a stepwise manner. In some embodiments, the stimulation intensity during the no-stimulation time period is about zero (e.g. the pulse generator is inactive) as is shown in FIG. 5. In certain embodiments, the stimulation intensity during the no-stimulation time period is substantially reduced from the maximum stimulation intensity applied during the stimulation time period. In certain embodiments, the stimulation intensity during the no-stimulation period is ramped down through at least two portions of the no-stimulation period. In certain embodiments, a decrease in stimulation intensity, if any, is approximately continuous over the entire no [symbol] -stimulation time period, rather than decreasing in single or multiple steps.

A single cycle of ramp-cycling therapy includes a stimulation time period and a no-stimulation time period. In some embodiments of the ramp-cycling technique, a single cycle may be repeated without changing any of the treatment parameters, the duty cycle parameters or the signal parameters of the original cycle. In certain embodiments the treatment parameters, and/or the duty cycle parameters and/or the signal parameters may be changed from cycle to cycle. In certain embodiments, a single cycle of ramp-cycling therapy comprises one to many suprathreshold periods and subthreshold periods.

Setting the stimulation signal parameters to particular values may inhibit substantial regain of lost weight for a relatively long time following the stimulation period. Indeed, weight and food intake may even continue to decline during the no-stimulation period, in which the stimulator is turned off. If the stimulation intensity is increased daily by about 20% over a period of several weeks until it is equal to about 8 times to about 10 times the signal intensity of muscle twitch threshold, and if the stimulator is subsequently turned off, then there is a period of about several days thereafter in which there is no rebound increase in weight or food intake.

In certain intermittent therapy treatment algorithm embodiments, ramp [symbol] -cycling therapy occurs during a period of about ten days to about two months. In this algorithm, the stimulation intensity during one portion of the stimulation time period is initiated and maintained at the muscle twitch threshold for about 24 hours. The stimulation intensity (current (mA) multiplied by pulse width (msec)) is increased by about 20% each day thereafter (i.e. during each subsequent portion of the simulation time period) until the stimulation intensity is about 8 times to about 10 times the muscle twitch threshold. After about 24 hours of stimulation at about 8 times to about 10 times the muscle twitch threshold, the stimulator is turned off during the no-stimulation time period of between about one-half day to about seven days. Utilizing a stimulation period of about 24 hours permits habituation of the muscle twitch, which reduces the discomfort experienced by the subject. Turning the stimulator off during the no stimulation time period on the order of days avoids a sustained increase in the MAP, reduces the likelihood that the subject develops a tolerance to the therapy, and preserves the stimulator's battery life.

In certain embodiments, a stimulation intensity increase of about 20% from one portion of the stimulation on period to the next portion is achieved by increasing the pulse width by about 20%. In certain embodiments, the stimulation intensity increase of about 20% is achieved by changing both the current and pulse width such that the product of the new values is about 20% greater that the product of the previous day's values for those parameters. In certain embodiments, the stimulation intensity increase of about 20% is achieved by increasing both the current and pulse width such that the product of the new values is about 20% greater that the product of the previous day's values for those parameters. In certain embodiments, the stimulation intensity increase of about 20% is achieved by increasing the current amplitude of the stimulation signal by about 20%.

In certain embodiments, the stimulation intensity increase of about 20% in a 24-hour period is achieved by an approximately continuous change in either the current amplitude, pulse width, or both. In certain embodiments, the stimulation signal intensity increase of about 20% in a 24 hour period is achieved by changing the current amplitude, pulse width, or both, at irregular intervals within each 24-hour period. In certain embodiments, the stimulation signal intensity increase of about 20% in a 24-hour period is achieved by changing the current amplitude, pulse width, or both, at regular intervals within each 24-hour period. In certain embodiments, the stimulation intensity increase of about 20% in a 24-hour period is achieved by changing the current amplitude, pulse width, or both, at regular intervals and in a stepwise manner within each 24-hour period. In certain embodiments, stimulation intensity increase of about 20% in a 24 hour period is achieved by changing the current amplitude, pulse width, or both, once during each 24-hour period. In certain embodiments, the stimulation intensity increase of about 20% in a 24 hour period is achieved by increasing the current amplitude once during each 24 hour period.

In certain embodiments, the stimulator is turned off in the cycle for between about 1 day and about 10 days. In certain embodiments, the stimulator is turned off for between about 1 day and about 5 days. In certain embodiments, the stimulator is turned off for about 3 days.

Certain embodiments include a method for treating a medical condition, the method comprising electrically activating a splanchnic nerve in a mammal for the stimulation time period, wherein the first time period comprises a plurality of consecutive intervals. During each of the plurality of consecutive intervals, the splanchnic nerve in the mammal is electrically activated according a stimulation pattern configured to result in net weight loss in the mammal during each interval. The stimulation pattern includes a signal on-time (on period or on-time) and a signal-off time (off period or off time) in a duty cycle. The on period includes a stimulation intensity and a frequency. In certain embodiments, the on period includes a suprathreshold period and a subthreshold period. The stimulation intensity includes a current amplitude and a pulse width. The method further includes reducing or ceasing the electrical activation of the splanchnic nerve for a no-stimulation time period, such that the mammal loses net weight during the no-stimulation period. In certain embodiments, the no-stimulation time period includes a subthreshold period.

In one embodiment, the duration of the stimulation time period is about ten days. In certain embodiments the duration of the stimulation time period is about 1 day to about 50 days. In certain embodiments the duration of the stimulation time period is about 4 hours to about 100 days. In some embodiments, there are ten consecutive intervals in the stimulation time period. In certain embodiments, there are about 3 intervals to about 50 intervals in the stimulation time period. In certain embodiments there are about 2 intervals to about 5000 intervals in the stimulation time period. In some embodiments, the duration of each consecutive interval is about 24 hours. In certain embodiments, the duration of each consecutive interval is about 12 hours to about 7 days. In certain embodiments, each consecutive interval is 1 minute to about 50 days.

In one embodiment, the duration of the on period is approximately equal to the duration of the interval, and the duration of the off period is approximately zero seconds. In some embodiments, the ratio of the on period to the off period is about 0.75 to about 1.5. In certain embodiments, the ratio is greater than about 0.75. In some embodiments, the ratio is greater than about 1.5. In certain embodiments, the ratio of the on period to the off period is greater than about 3. In certain embodiments, the ratio of the on period to the off period is about 0.75 or less, while in certain embodiments the ratio is about 0.5 or less. In certain embodiments, the ratio of the on period to the off period is about 0.3 or less. In certain embodiments, the on period is about two minutes or less. In some embodiments, the on period is about one minute or less. In certain embodiments, the on period is about one minute or less, and the off period is about one minute or more. In some embodiments the on period is greater than about 15 seconds but in certain embodiments, the on-time is greater than about 30 seconds.

In one embodiment, the duration of the suprathreshold period is approximately equal to the duration of the interval, and the duration of the subthreshold period is approximately zero seconds. In some embodiments, the ratio of the suprathreshold period to the subthreshold period is about 0.75 to about 1.5. In certain embodiments, the ratio is greater than about 0.75. In some embodiments, the ratio is greater than about 1.5. In certain embodiments, the ratio of the suprathreshold period to the subthreshold period is greater than about 3. In certain embodiments, the ratio of the suprathreshold period to the subthreshold period is about 0.75 or less, while in certain embodiments the ratio is about 0.5 or less. In certain embodiments, the ratio of the suprathreshold period to the subthreshold period is about 0.3 or less. In certain embodiments, the suprathreshold period is about two minutes or less. In some embodiments, the suprathreshold period is about one minute or less. In certain embodiments, the suprathreshold period is about one minute or less, and the subthreshold period is about one minute or more. In some embodiments the suprathreshold period is greater than about 15 seconds but in certain embodiments, the on-time is greater than about 30 seconds.

In some embodiments the combined on period and off period cycle is repeated continuously within the interval. In certain embodiments the combined on period and off period cycle is repeated intermittently within the interval. In certain embodiments, the combined on period and off period cycle is repeated irregularly within the interval. In some embodiments the combined suprathreshold period and subthreshold period cycle is repeated continuously within the interval. In certain embodiments the combined suprathreshold period and subthreshold period cycle is repeated intermittently within the interval. In certain embodiments, the combined suprathreshold period and subthreshold period cycle is repeated irregularly within the interval. In some embodiments, the frequency of the stimulation signal is about 15 Hz or greater to minimize skeletal twitching. In some embodiments the frequency of the stimulation signal is about 20 Hz or greater. In some embodiments the frequency of the stimulation signal is about 30 Hz or greater. In some embodiments, the frequency is varied within each interval, but in certain embodiments the frequency remains constant within each interval. In some embodiments the frequency is varied from interval to interval, but in certain embodiments the frequency remains constant.

In some embodiments the stimulation intensity of the signal is varied within each interval during the stimulation time period, but in certain embodiments, the stimulation intensity remains constant within each interval during the stimulation time period. In some embodiments the stimulation intensity is varied from interval to interval during the stimulation time period. In some embodiments the stimulation signal intensity is increased from interval to interval during the stimulation time period. In some embodiments the stimulation intensity of the first interval during the stimulation time period is set at about the muscle twitch threshold. In some embodiments the first interval is set below the muscle twitch threshold, while in certain embodiments the first interval is set above the muscle twitch threshold.

In some embodiments the stimulation intensity is increased by about 20% from interval to interval during the stimulation time period. In some embodiments the stimulation intensity is increased by about 15% to about 25% from interval to interval. In certain embodiments, the stimulation intensity is increased by about 1% to about 15% from interval to interval. In certain embodiments, the stimulation intensity is increased by about 25% to about 40% from interval to interval. In certain embodiments the stimulation intensity is increased by about 40% to about 100% from interval to interval.

In some embodiments the stimulation signal intensity is varied by changing the current amplitude. In some embodiments the stimulation intensity is varied by changing the pulse width. In some embodiments, the stimulation signal intensity is varied by changing the electrical potential. In some embodiments the stimulation intensity is varied by changing any combination of the current amplitude, the pulse width, and the electrical potential or voltage.

In some embodiments the no-stimulation time period is about 4 days. In some embodiments the no-stimulation time period is about 1 day to about 7 days. In some embodiments the no-stimulation time period is about 18 hours to about 10 days. In some embodiments the no-stimulation time period is about 1 hour to about 50 days. In some embodiments the no-stimulation time period is more than about 50 days. In some embodiments the no-stimulation time period is less than about 1 day. In some embodiments the no-stimulation time period is less than about 6 hours. In certain embodiments, the second time period is less than about 1 hour.

The following three ramp-cycling algorithm embodiments were tested for their efficacy. Each experiment lasted for 28 days. The first algorithm used daily, stepwise increases in the current amplitude of the stimulation signal to increase the stimulation intensity during the stimulation time period. The stimulation intensity was so increased for 9 consecutive days within the stimulation time period. On the 10th day, the no-stimulation time period began. During the no stimulation time period the stimulator was turned off and remained off for 4 days. The above cycle was then repeated.

The second of the three ramp-cycling algorithms used daily, stepwise increases in the current amplitude to increase the stimulation intensity during the stimulation time period. The stimulation intensity was so increased for 9 consecutive days. On the 10th day, the no-stimulation time period began. During the no-stimulation time period the stimulator was turned off and remained off for 3 days. That cycle was then repeated.

The third of the three ramp-cycling algorithms used daily, stepwise increases in the current amplitude to increase the stimulation intensity during the stimulation time period. The stimulation intensity was so increased for 9 consecutive days. On the 10th day, the no-stimulation time period began. In this case, the stimulation intensity was reduced to a non-zero threshold value during the no-stimulation time period. The cycle was then repeated. This algorithm did not contain a no-stimulation time period where the stimulator was turned off.

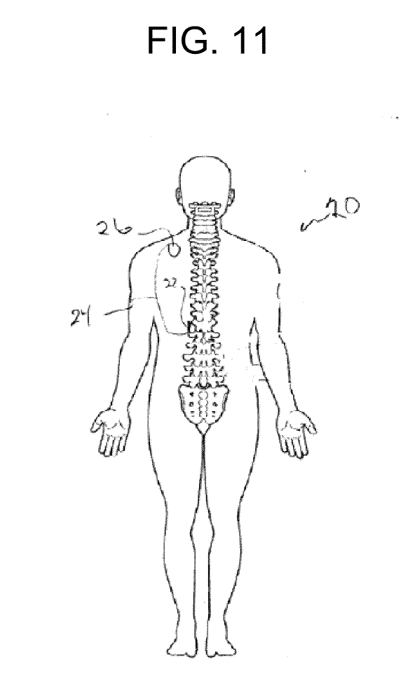

FIG. 11 illustrates a schematic view of an IPG implanted within a human body. The IPG can be a neurostimulator which may be similar in some respects to existing neurostimulators. In this illustration, the IPG has an output coupled to a nerve cuff which is positioned over the Greater Splanchnic Nerve (GSN). Various electrodes may be used in various embodiments, including but not limited to cuff electrodes, patch electrodes, monopolar, bipolar, tripolar, and quadrapolar electrodes. In some embodiments, the housing of the IPG can serve and one of the electrodes.

In some embodiments, the current supplied can vary in current intensity from about 0 mA to about 10 mA, in increments. Some IPGs output pulse trains having a number of pulses having a frequency which can vary from about 1 Hz to about 40 Hz. Some devices allow for the ramping of current and/or frequency. The IPG shown has a “SP” providing a “Set Point” as input, for the desired blood pressure. In practice, this BP would likely be provided at the time of implantation, and may be provided through telemetry in many embodiments.

The IPG illustrated also includes an input for receiving the blood pressure signal from a BP sensor which is positioned near or within an artery. The signal can be transmitted electronically or optically, in various embodiments.

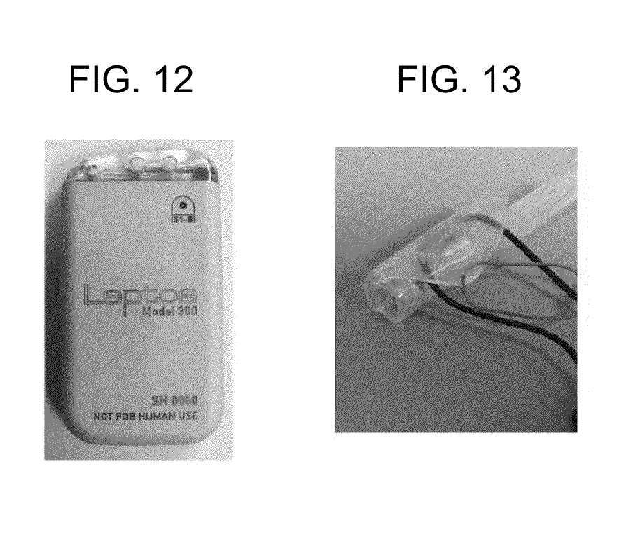

FIG. 12 illustrates the general nature of an IPG than may be used to stimulate a nerve is some embodiments of the invention. The IPG shown is a hermetically sealed device having a titanium housing having stimulation circuitry and optionally sensing circuitry within. An IPG according to the present invention would have an input for receiving a BP signal as well, for example, in the header.

FIG. 13 illustrates on electrode that may be used in some embodiments of the invention. The electrode shown is a tripolar cuff electrode.

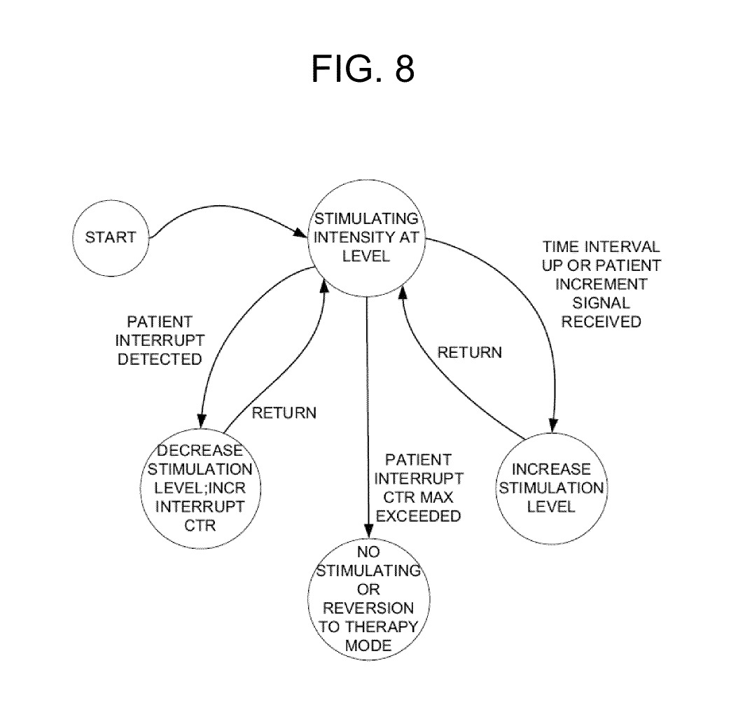

FIG. 8 illustrates on example of logic that can be executed in an IPG. The various parameters can be downloaded to the IPG device using a clinical programmer or a patient programmer device, through an RF or inductively coupled communication link. These communication links are well known to those skilled in the art. The logic can be executed in a programmable microcontroller, or programmable logic device, and other technologies well known to those skilled in the art.

The IPG can start in a start state, where the IPG may be idling, waiting for a command to being stimulating, or stimulating. Upon reception of a start signal, the IPG can begin stimulating using a current maximum stimulation current. The stimulation therapy may include ramp ups, ramp downs, or other dynamic algorithms. These ramps may be on the order of a few seconds, half a minute or a minute, and on the order of hours, depending on the therapy and the various reasons for the ramps. The ramps often ramp current up to a maximum, e.g. a ramp to the current maximum current over 30 minutes, during which time the stimulation pulse increase in their current amplitudes over a 30 minute period. In some embodiments, the pulse are delivered in pulse trains to form a “dose” that may have a duration ranging from several seconds to an hour or more, where the pulse trains may or may not be uninterrupted over the course of the dose, depending on the embodiment.

In one example, intended for illustration, not limitation, several doses are delivered during the day, separated by inter-dose periods. The maximum current intensity, at the top of the dose, may be set at a particular value for the day. In one example, the maximum current intensity level is initially started out at 0.5 mA, and held at that level for one day. The next day the current is programmed to increase by 0.5 mA, to a value of 1.0 mA. This may continue for around 7 days or a week, whereupon the stimulation current drops to zero or a non-zero sub-therapeutic sub-threshold. After 2-3 days, in this example, the week-long pattern occurs again. Therefore, in one example, the maximum current increases by 0.5 mA the first day, in several doses over the day. One the second day, the 0.5 mA current maximum is “challenged” or urged upward by the additional current. In some therapies, this increase in stimulation intensities can be increased over the course of a day.

While not wishing to be bound by theory, one purpose of the ramp is to avoid habituation by stimulating nerves either smaller in diameter and/or located more deeply within a nerve bundle. By recruiting new nerve fibers, there is new stimulation even if the originally recruited nerve fibers are temporarily exhausted. Recruiting more nerve fibers may or may not also mimic normal stimulation patterns, and promote the more desired response. In addition, the higher stimulation intensity may be required to elicit the desired response. In one example, efferent stimulation may be required to affect a desired therapeutic response, and that may require 3 mA. If 3 mA is utilized at the very begging, the sudden stimulation at this level may provoke a feeling of discomfort in the patient. If the threshold of such discomfort can be urged upward by a gradual increase in stimulation current, then the ultimate desired stimulation current can be attained through such nudging or challenging of the threshold.

The challenging can have a number of parameters to be used in determining how to how to configure the challenging logic, what signals to output, and when to end the challenge mode. The end of the challenge mode may also be referred to as the end of the test mode. The challenging may be considered to have a base current level, an amount to increase by, a maximum current level, and a time period to elapse between increases in the maximum current, all as parameters which may be downloaded or set by a computer during placement and/or in the treating physician's office. Such parameters may also be modified using a patient programmer which may be used by the patient to modify the parameters at home.

In the example given, the automatic increase in stimulation current may create a perception of discomfort in the patient. It may be desirable for the IPG to retreat to the previous maximum current level, at least for a while. In the example where the maximum current is increased by 0.5 mA each day, the most recent increase of 0.5 mA may be reversed, and the previous maximum current used for the remainder of the day. The next day, the maximum current may be increased by 0.5 mA again, with hopefully no discomfort.

If the patient still does not tolerate the increased stimulation current, the patient may again request that the increase be undone. In some embodiments, there is a limit to the number of patient discomfort indications that can be accepted before further changes are made. In one such example, after a set maximum number of discomfort indications are made, no further increases in maximum current are made. In another such example, the stimulation is stopped altogether.

FIG. 8 shows that after the START state, the STIMULATING AT LEVEL state begins. This usually refers to stimulating at a maximum level, for example, the maximum current at the top of a dose ramp for that day. After the time interval is up (e.g. 1 day) or a certain clock time of day (e.g. 6 AM), the INCREASE STIMULATION LEVEL state may be entered. In one example, the maximum current is increased by 0.5 mA, and the STIMULATING AT LEVEL state is returned to.

If the patient feels discomfort, the patient may indicate this to the IPG via a patient signal or a patient interrupt as indicated on FIG. 8. Upon sensing this signal, the IPG can enter the DECREASE STIMULATION LEVEL state to decrease the maximum stimulation level by a decrease in current amount. This decrease in current amount may be the same amount as the increase, less than the increase, or greater than the increase, in various embodiments. In addition to decreasing the maximum current, the state can increment an interrupt counter, indicated as INCR INTERRUPT CTR in FIG. 8. In this way, the number of indications of discomfort can be tracked and utilized in the end of test decision criteria, in some embodiments. The STIMULATING AT LEVEL state is returned to.

When the maximum number of patient interrupts is exceeded, the NO STIMULATING state may be entered, and stimulating stopped IN SOME EMBODIMENTS. In other embodiments, a REVERSION TO THERAPY MODE is entered, in which the stimulation is not stopped, but further automatic increases in maximum stimulation current are no longer performed. In some embodiments, therapy mode is entered using the last tolerated stimulation level.

The patient interrupts may be performed in various ways in various embodiments. In some embodiments, a patient programmer may be used, intended to be used by the patient, and often having fewer features than a clinical programmer used by a medical professional. In some embodiments, a magnet may be held in place over an implanted sensor coupled to, within, or part of the IPG. The magnetic sensor may be a reed switch or functional equivalent, e.g. a Hall effect device. In some embodiments, a specific signal must be received, for example the magnet held in place for 5-10 seconds, followed by removal for 5-10 seconds, followed by more magnet application for an additional 5-10 seconds. In some methods, further indications of discomfort are ignored for what is effectively a refractory period e.g. at least 30 minutes after the first indication of discomfort is made. In some methods, each indication of discomfort results in a further decrease in maximum stimulation current.

In one method, the magnet serves as the patient interrupt device, similar to or the same as a function of the patient programmer device. In one method, holding the magnet over the IPG instructs the IPG to stop stimulation for as long as the magnet is in place and for a certain time period thereafter. Holding the magnet in place also serves to decrease the maximum stimulation back to the previous value, and to increment the counter of patient interrupts. In one method, the magnet can be used several times in one day with effect, but can only increase the count of the number of patient interrupts once per day.

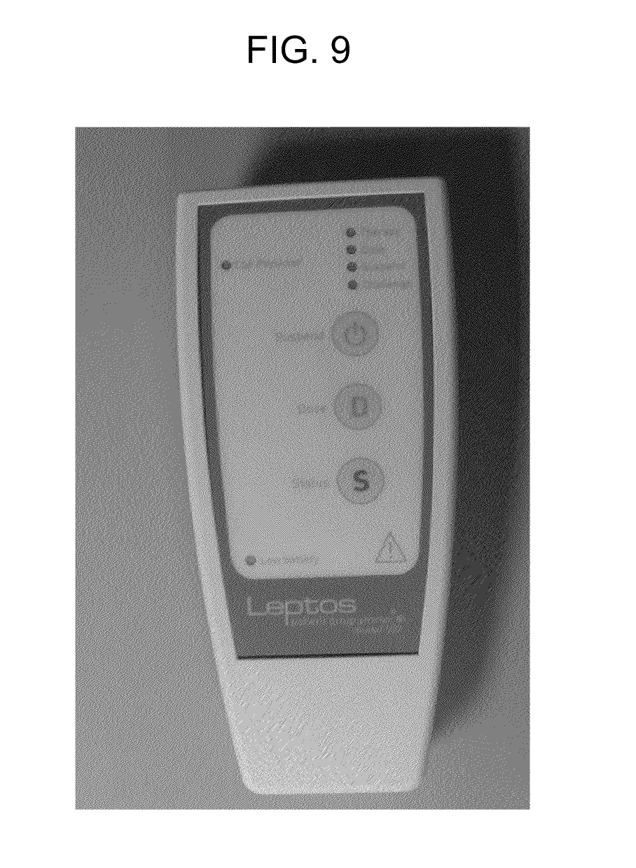

FIG. 9 illustrates one handheld patient programmer device according to present invention. This device can communicate with the IPG using telemetry through inductive coupling.

The device has three buttons which may be pressed by the patient. The lower button is the STATUS button, which may be used to query the IPG to transmit the device status, which is indicated by the 4 upper status lights and also the upper left CALL PHYSICIAN light. The middle button is the DOSE button, which instructs the IPG to deliver a dose of therapy. This dose, in one embodiment, is a dose having a profile, length, frequency, and maximum current set in the IPG by a medical professional. As long as the dose is being delivered, the DOSE light will be the status returned by the IPG. The SUSPEND button may be pressed, in one embodiment, to serve the same function as the magnet placement. The SUSPEND light will show a suspend status for a certain time period e.g. 30 minutes after the IPG was instructed to suspend, either by the magnet or the patient programmer.

In one use of the IPG, the IPG is programmed in the physician's office to start out in the challenge mode, meaning that the IPG is to determine the maximum tolerated stimulation current for the patient. In this use example, a number of stimulation doses are given during the day, having a programmed profile/shape, frequency, etc. The maximum current for each dose will be increased each day in this example, as long as the IPG is in challenge mode. The CHALLENGE light on the hand held unit will indicate this mode.

In this example, if the patient has felt discomfort, and has used the magnet or the SUSPEND button on the patient programmer on three different days, then the IPG will use the maximum tolerated maximum current and enter the therapy mode, indicated by the THERAPY light on the patient programmer. In therapy mode, the doses will be delivered, using the maximum tolerated current, but that maximum tolerated current will not be increased any more.

The CALL PHYSICIAN light indicates a fault or other condition in the IPG requiring a call to the physician. The LOW BATTERY light indicates a low battery level in the patient programmer.

In one embodiment, holding the patient programmer sufficiently close to the IPG while pushing the suspend button will continue to act as though the suspend button is continually depressed for a long as there is communication between the IPG and the patient programmer.

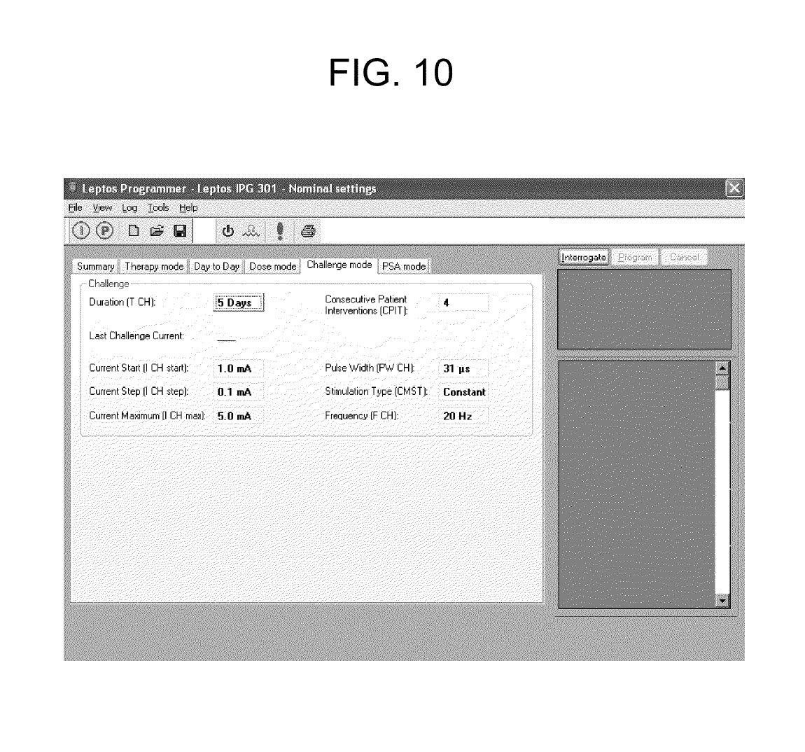

FIG. 10 shows the Challenge Mode Screen on a Clinical programming device, having the various parameters described herein, although using different nomenclature, and some fields not necessary for understanding the invention to be claimed. A duration parameter field is shown, having a value of 5 days, indicating the challenge mode will last for up to 5 days before reverting to the therapy mode. A current start parameter is shown, having a 1.0 mA value, the first maximum stimulation current to be tried. A current step parameter is shown, having a 0.1 mA value, the amount of increase to be added to the maximum current value in each increment. A pulse width parameter is shown 31 usec. A simulation type field is shown as well, shown as constant as opposed to circadian, which can vary the stimulation at night. The number of consecutive patient interventions is shown as 4, indicating that after 4 patient interventions, the challenge mode will change to therapy mode. A last challenge current field is shown as well, as blank, as this can be downloaded from an IPG.

What is claimed:

1. A method of treating insulin resistance comprising:
selecting a subject having insulin resistance;
initiating an electrical stimulation pattern wherein the electrical stimulation pattern stimulates the splanchnic nerve and wherein the electrical stimulation pattern achieves a maximum tolerable stimulation intensity comprising the characteristics of a pulse width, frequency and current and wherein upon an increment event the maximum tolerable stimulation intensity is increased by an increase amount;
upon receiving a patient initiated signal, interrupting the electrical stimulation pattern using a patient intervention device (PID), where upon interruption the maximum tolerable stimulation intensity is decreased by a decrease amount, the decrease lessens the sensation caused by the electrical stimulation pattern resulting in a new maximum tolerable stimulation intensity; and
automatically initiating the electrical stimulation pattern, where upon an increment event the maximum tolerable stimulation intensity is increased.

2. The method of claim 1, in which the increment event is selected from the passage of a time period, the occurrence of a time event, the passage of a time period without receiving the patient initiated signal or combinations thereof.

3. The method of claim 1, wherein the PID is selected from the group consisting of a magnet, a patient programmer, or a sound signal emitter.

4. The method of claim 1, in which the maximum tolerable stimulation intensity level cannot be increased above an intensity limit.

5. The method of claim 1, further comprising incrementing a counter upon receiving the patient initiated signal and discontinuing the stimulation when the counter exceeds a counter limit.

6. The method of claim 1, further comprising upon receiving a patient initiated signal pausing stimulation.

7. The method of claim 1, further comprising upon receiving a patient initiated signal increasing stimulation intensity.

8. The method according to claim 1, wherein upon receiving a patient initiated signal a biomarker reading is taken.

9. The method according to claim 1, further comprising periodically measuring biomarkers.

10. The method according to claim 1, wherein selecting a subject having insulin resistance comprises measuring at least one of HbA1c, fasting glucose or fasting insulin.

11. A method for reducing central adiposity comprising:
electrically stimulating the splanchnic nerve of a patient using a stimulation pattern comprising a stimulation intensity wherein the stimulation intensity comprises a pulse width, frequency and current and wherein the stimulation pattern comprises periodically increasing the stimulation intensity upon occurrence of an increment event;
interrupting the stimulation pattern with a patient intervention device, wherein upon interruption the stimulation intensity is decreased; and
re-establishing the stimulation pattern wherein the stimulation intensity is periodically increased, and wherein central adiposity is decreased.

12. The method according to claim 11, wherein central adiposity is measured by at least one of dual-energy x-ray absorptiometry (DEXA), circumference measurement, or computed axial tomography (CT).

13. The method of claim 11, in which the increment event is selected from the passage of a time period, the occurrence of a time event, the passage of a time period without receiving the patient initiated signal or combinations thereof.

14. The method of claim 11, in which the maximum tolerable stimulation intensity level cannot be increased above an intensity limit.

15. The method of claim 11, further comprising incrementing a counter upon receiving the patient initiated signal and discontinuing the stimulation when the counter exceeds a counter limit.

16. The method of claim 11, further comprising upon receiving a patient initiated signal pausing stimulation.

17. The method of claim 11, further comprising upon receiving a patient initiated signal increasing stimulation intensity.

18. The method according to claim 11, wherein upon receiving a patient initiated signal a biomarker reading is taken.

19. The method according to claim 11, further comprising periodically measuring biomarkers.
